(19) United States
(12) Patent Application Publication
(10) Pub. No.: US 2021/0395694 A1
(43) Pub. Date: Dec. 23, 2021

(54) METHOD FOR PROMOTING PRODUCTION OF EXOSOMES AND/OR EXTRACELLULAR VESICLES

(71) Applicant: ExoCoBio Inc., Seoul (KR)

(72) Inventors: Byong Seung CHO, Gunpo-si (KR); Dae Hyun HA, Seoul (KR)

(73) Assignee: ExoCoBio Inc., Seoul (KR)

(21) Appl. No.: 17/465,022

(22) Filed: Sep. 2, 2021

Related U.S. Application Data
(63) Continuation of application No. PCT/KR2020/007798, filed on Jun. 17, 2020.

(30) Foreign Application Priority Data
Sep. 18, 2019 (KR) 10-2019-0114456
May 31, 2020 (KR) 10-2020-0065539

(51) Int. Cl. C12N 5/0775

(52) U.S. Cl. CPC C12N 5/0667; C12N 2500/90; C12N 2500/38

(57) ABSTRACT

A method for promoting production of exosomes and/or extracellular vesicles is provided. The method includes culturing animal cells in a medium containing ascorbic acid, an analogue thereof, or a derivative thereof. The method is able to enhance the productivity of exosomes and/or extracellular vesicles secreted or released per cell or cell-derived conditioned medium.

### CROSS REFERENCE

This application is a Bypass Continuation of International Application No. PCT/KR2020/007798 filed Jun. 17, 2020, claiming priority based on Korean Patent Application No. 10-2019-0114456 filed Sep. 18, 2019 and Korean Patent Application No. 10-2020-0065539 filed May 31, 2020, the entire contents of which are incorporated herein by reference.

### TECHNICAL FIELD The present invention relates to a method for promoting production of exosomes and/or extracellular vesicles.

In addition, the present invention relates to a medium for promoting production of exosomes and/or extracellular vesicles.

### BACKGROUND ART

Recently, there have been reports that cell secretomes contain various bioactive molecules that regulate cellular behaviors. In particular, cell secretomes contain ‘exosome’ or ‘extracellular vesicle’ that has intercellular signaling functions, and thus studies on the components and functions thereof have been actively conducted.

Cells shed various membraneous vesicles to their extracellular environment, and these released vesicles are usually called extracellular vesicles (EVs). The EV is also called cell membrane-derived vesicle, ectosome, shedding vesicle, microparticle, exosome, etc., and is also used discriminately from exosome in some cases.

Exosome is a vesicle of tens to hundreds of nanometers in size, which comprises a phospholipid bilayer membrane having the same structure as that of the cell membrane. This exosome contains proteins, nucleic acids (mRNA, miRNA, etc.) and the like which are called exosome cargo. It is known that exosome cargo includes a wide range of signaling factors, and these signaling factors are specific for cell types and regulated differently depending on secretory cells' environment. It is known that exosome is an intercellular signaling mediator secreted by cells, and various cellular signals transmitted through it regulate cellular behaviors, including the activation, growth, migration, differentiation, dedifferentiation, apoptosis, and necrosis of target cells. Exosome contains specific genetic materials and bioactive factors depending on the nature and state of cells from which the exosome was derived. Exosome derived from proliferating stem cells regulates cell behaviors such as cell migration, proliferation and differentiation, and recapitulates the characteristics of stem cells involved in tissue regeneration (Nature Review Immunology 2002 (2) 569-579).

That is, exosomes called “avatars” of cells contain bioactive factors such as growth factors, similar to cells, and serve as carriers that transmit bioactive factors between cells, that is, serve to mediate cell-to-cell communication. Exosomes are known to be released not only from animal cells such as stem cells, immune cells, fibroblasts and cancer cells, but also from cells of various organisms such as plants, bacteria, fungi, and algae. Conventional techniques for isolating exosomes or extracellular vesicles include ultracentrifugation, density gradient centrifugation, ultrafiltration, tangential flow filtration (TFF), size exclusion chromatography, ion-exchange chromatography, immunoaffinity capture, microfluidics-based isolation, exosome precipitation, total exosome isolation kit, polymer based precipitation, and the like.

However, although various methods for isolating exosomes and/or extracellular vesicles as described above have been proposed, continuous improvement can be made in the technical field related to the production of exosomes and/or extracellular vesicles, similar to other technical fields. For example, there is a need to provide a new technique for producing exosomes and/or extracellular vesicles, which can enhance the productivity of exosomes and/or extracellular vesicles secreted or released per cell (or cell-derived conditioned medium).

Meanwhile, it is to be understood that the matters described as the background art are intended merely to aid in the understanding of the background of the present invention and are not admitted as prior art against the present invention.

### SUMMARY OF INVENTION

An object of the present invention is to provide a method for promoting production of exosomes and/or extracellular vesicles.

Another object of the present invention is to provide a medium for promoting production of exosomes and/or extracellular vesicles.

However, the objects of the present invention as described above are illustrative and the scope of the present invention is not limited thereby. In addition, other objects and advantages of the present invention will be more apparent from the following description, the appended claims and the accompanying drawings.

### DETAILED DESCRIPTION OF INVENTION

The present inventors have conducted intensive studies on a method for producing exosomes and/or extracellular vesicles that can enhance the productivity of exosomes and/or extracellular vesicles, and as a result, have found that when cells are cultured in a medium containing ascorbic acid, an analogue thereof, or a derivative thereof, the productivity of exosomes and/or extracellular vesicles secreted or released per cell (or cell-derived conditioned medium) is enhanced, thereby completing the present invention.

The present invention provides a method for promoting production of exosomes and/or extracellular vesicles or for enhancing productivity thereof, the method comprising culturing animal cells in a medium containing ascorbic acid, an analogue thereof, or a derivative thereof.

As used herein, the term “extracellular vesicles (EVs)” is generally meant to encompass membrane vesicles, ectosomes, shedding vesicles, microparticles, or equivalents thereto. Depending on the isolation environment, conditions and methods, the term “extracellular vesicles” may have the same meaning as the term “exosomes”, and may also be meant to include nanovesicles that have the same or similar size as exosomes, but have a composition which differs from that of exosomes. Meanwhile, the term “exosomes” as used in relation to promotion of production or enhancement of productivity is meant to encompass the aforesaid extracellular vesicles.

As used herein, the term “exosomes” refers to vesicles of tens to hundreds of nanometers in size (preferably, about 30 to 200 nm), which comprise a phospholipid bilayer membrane having the same structure as that of the cell membrane (however, the particle size of exosomes is variable depending on the type of cell from which the exosomes are isolated, an isolation method and a measurement method) (Vasiliy S. Chernyshev et al., “Size and shape characterization of hydrated and desiccated exosomes”, Anal Bioanal Chem, (2015) DOI 10.1007/s00216-015-8535-3). These exosomes contain proteins, nucleic acids (mRNA, miRNA, etc.) and the like which are called exosome cargo. It is known that exosome cargo includes a wide range of signaling factors, and these signaling factors are specific for cell types and regulated differently depending on secretory cells' environment. It is known that exosomes are intercellular signaling mediators secreted by cells, and various cellular signals transmitted through them regulate cellular behaviors, including the activation, growth, migration, differentiation, dedifferentiation, apoptosis, and necrosis of target cells.

Meanwhile, the term “exosomes” as used herein is intended to include all vesicles (e.g., exosome-like vesicles) which are secreted from animal cells and released into extracellular spaces, and have a nano-sized vesicle structure and a composition similar to that of exosomes.

In the present invention, the type of animal cells from which exosomes and/or extracellular vesicles are derived is not limited. As an example not limiting the scope of the present invention, the animal cells may be stem cells or immune cells. The stem cells may be embryonic stem cells, induced pluripotent stem cells (iPSCs), adult stem cells, embryonic stem cell-derived mesenchymal stem cells, or induced pluripotent stem cell-derived mesenchymal stem cells. The immune cells may be T cells, B cells, NK cells, cytotoxic T cells, dendritic cells or macrophages.

As an example not limiting the scope of the present invention, the adult stem cells may be at least one type of adult stem cells selected from the group consisting of mesenchymal stem cells, human tissue-derived mesenchymal stromal cells, human tissue-derived mesenchymal stem cells, and pluripotent stem cells. The mesenchymal stem cells may be mesenchymal stem cells derived from at least one tissue selected from the group consisting of umbilical cord, umbilical cord blood, bone marrow, adipose tissue, muscle, nerve, skin, amniotic membrane, Wharton's jelly, and placenta. Preferably, the adult stem cells may be mesenchymal stem cells, for example, adipose-, bone marrow-, umbilical cord- or umbilical cord blood-derived stem cells, more preferably adipose-derived stem cells. The stem cells or the immune cells are not limited to the kind thereof, as long as they do not pose a risk of infection with a pathogen and do not cause immune rejection, but may preferably be human stem cells or human immune cells.

However, it is of course possible to use various animal cells that are being used in the art or may be used in the future, as long as they do not cause adverse effects on the human body. For example, it is also possible to use HEK293 cells or HEK293T cells. Thus, it is to be understood that the adipose-derived stem cell used in the Examples described later is an example of animal cells that may be used in the present invention, and the present invention is not limited thereto.

As used herein, the term “ascorbic acid, an analogue thereof, or a derivative thereof” refers to, for example, ascorbic acid, ascorbic acid-2-glucoside (AA-2G), ascorbic acid-2-sulfate (AA-2S), ascorbic acid-2-phosphate (AA-2P), magnesium ascorbyl phosphate, ascorbyl palmitate, retinyl ascorbate, tetrahexyldecyl ascorbate, sodium ascorbate, calcium ascorbate, polyethoxylated ascorbic acid, ethyl ascorbic acid, aminopropyl ascorbyl phosphate, and/or 3-ascorbyl carbonyl dipeptide-17. However, it is to be understood that the above-described ascorbic acid, analogue thereof, or derivative thereof is an example of ascorbic acid, analogues thereof, or derivatives thereof that may be used in the present invention, and the present invention is not limited thereto.

As used herein, the term “pre-treated” means culturing animal cells in a medium containing ascorbic acid, an analogue thereof, or a derivative thereof, and the term “pre-treated exosomes and/or extracellular vesicles” refers to exosomes and/or extracellular vesicles obtained by culturing animal cells in a medium containing ascorbic acid, an analogue thereof, or a derivative thereof The term “untreated” means culturing animal cells in a medium that does not contain ascorbic acid, an analogue thereof, or a derivative thereof The term “untreated exosomes and/or extracellular vesicles or untreated control” refers to exosomes and/or extracellular vesicles obtained without being treated with ascorbic acid, an analogue thereof, or a derivative thereof, in any of a cell culture step and an exosome isolation step.

The present invention provides a method for promoting production of exosomes and/or extracellular vesicles, the method comprising culturing animal cells in a medium containing ascorbic acid, an analogue thereof, or a derivative thereof.

The method for promoting production of exosomes and/or extracellular vesicles according to one embodiment of the present invention may further comprise culturing the animal cells in a medium containing ascorbic acid, an analogue thereof, or a derivative thereof and fetal bovine serum (FBS). The fetal bovine serum may be one from which FBS-derived exosomes and extracellular vesicles have been removed.

The method for promoting production of exosomes and/or extracellular vesicles according to one embodiment of the present invention may further comprise culturing the animal cells in a serum-free medium containing ascorbic acid, an analogue thereof, or a derivative thereof.

In the method for promoting production of exosomes and/or extracellular vesicles according to one embodiment according to the present invention, the concentration of the ascorbic acid, analogue thereof, or derivative thereof may be about 1 μg/mL to 300 μg/mL, about 10 μg/mL to 100 μg/mL, about 10 μg/mL to 200 μg/mL, about 10 μg/mL to 300 μg/mL, about 50 μg/mL to 300 μg/mL, about 50 μg/mL to 200 μg/mL, about 50 μg/mL to 100 μg/mL, about 5 μg/mL to 100 μg/mL, or about 5 μg/mL to 200 μg/mL.

In the method for promoting production of exosomes and/or extracellular vesicles of one embodiment according to the present invention, the amount of exosomes and/or extracellular vesicles produced may be defined as the content of exosomes and/or extracellular vesicles (i.e., the tetraspanin content, for example, the CD63 content) per cell (or cell-derived conditioned medium), or the number of exosome and/or extracellular vesicle particles per cell (or cell-derived conditioned medium).

The present invention provides a medium for promoting production of animal cell-derived exosomes and/or extracellular vesicles, the medium containing ascorbic acid, an analogue thereof, or a derivative thereof.

In the medium for promoting production of animal cell-derived exosomes and/or extracellular vesicles according to one embodiment of the present invention, the concentration of the ascorbic acid, analogue thereof, or derivative thereof may be about 1 μg/mL to 300 μg/mL, about 10 μg/mL to 100 μg/mL, about 10 μg/mL to 200 μg/mL, about 10 μg/mL to 300 μg/mL, about 50 μg/mL to 300 μg/mL, about 50 μg/mL to 200 μg/mL, about 50 μg/mL to 100 μg/mL, about 5 μg/mL to 100 μg/mL, or about 5 μg/mL to 200 μg/mL.

### ADVANTAGEOUS EFFECTS

The present invention is able to enhance the productivity of exosomes and/or extracellular vesicles secreted or released per cell (or cell-derived conditioned medium). Therefore, the method for promoting production of exosomes and/or extracellular vesicles according to the present invention has advantages that it can economically and efficiently produce exosomes and/or extracellular vesicles, which can be utilized commercially and/or clinically, in high yield, and in particular, it can produce a large amount of exosomes and/or extracellular vesicles as compared with conventional methods.

It should be understood that the scope of the present invention is not limited to the aforementioned effects.

### BRIEF DESCRIPTION OF DRAWINGS

FIG. 1A is a graph showing the particle size distribution and the number of particles obtained by performing nanoparticle tracking analysis (NTA) for exosomes obtained after culturing stem cells in a medium not containing ascorbic acid, an analogue thereof, or a derivative thereof (indicated as “0 μg/mL”).

FIG. 1B is a graph showing the particle size distribution and the number of particles obtained by performing nanoparticle tracking analysis (NTA) for exosomes obtained after pre-treating and culturing stem cells in a medium containing ascorbic acid, an analogue thereof, or a derivative thereof (indicated as “30 μg/mL”).

FIG. 1C is a graph showing the particle size distribution and the number of particles obtained by performing nanoparticle tracking analysis (NTA) for exosomes obtained after pre-treating and culturing stem cells in a medium containing ascorbic acid, an analogue thereof, or a derivative thereof (indicated as “100 μg/mL”).

FIG. 2 is a comparative graph showing that when stem cells were pre-treated and cultured in a medium containing ascorbic acid, an analogue thereof, or a derivative thereof, the number of exosome particles per mL (i.e., exosome productivity) remarkably increased compared to that in an untreated control group, i.e., not treated with ascorbic acid, an analogue thereof, or a derivative thereof

FIG. 3 is a comparative graph showing that when stem cells were pre-treated and cultured in a medium containing ascorbic acid, an analogue thereof, or a derivative thereof, the exosome content (i.e., CD63 content) per mL remarkably increased compared to that in an untreated control group, i.e., not treated with ascorbic acid, an analogue thereof, or a derivative thereof.

### EXAMPLES

Hereinafter, the present invention will be described in more detail with reference to the following examples. However, the following examples are only to illustrate the present invention and are not intended to limit or restrict the scope of the present invention. Those that can be easily inferred by those skilled in the art from the detailed description and examples of the present invention are interpreted as falling within the scope of the present invention. References referred to in the present invention are incorporated herein by reference.

Throughout the present specification, it is to be understood that, when any part is referred to as “comprising” any component, it does not exclude other components, but may further include other components, unless otherwise specified.

### Example 1

### Cell Culture

Human adipose-derived stem cells were suspended in a-MEM culture medium containing 10% fetal bovine serum (FBS), 100 units/mL penicillin and 100 μg/mL streptomycin, and then seeded into a 6-well plate at a density of 6,000 cells/cm² and cultured in an incubator at 37° C. under 5% CO₂. For ascorbic acid-treated groups, 24 hours after seeding, ascorbic acid was added to the culture medium at a concentration of 10 μg/mL, 30 μg/mL and 100 μg/mL, respectively.

In all of the ascorbic acid-treated groups and the untreated group, when the adipose-derived stem cells reached a confluency of 80% or more, the culture medium was replaced with α-MEM medium containing 100 units/mL penicillin and 100 μg/mL streptomycin (hereinafter referred to as “serum-free medium” or “SFM”), or α-MEM medium containing 2% FBS from which FBS-derived exosomes and extracellular vesicles have been removed, 100 units/mL penicillin and 100 μg/mL streptomycin (hereinafter referred to as “extracellular vesicles-depleted medium” or “EDM”). In the ascorbic acid-treated groups, ascorbic acid was added to each of the serum-free medium and the extracellular vesicles-depleted medium at a concentration of 10 μg/mL, 30 μg/mL and 100 μg/mL, respectively. Thereafter, the cells were cultured for 24 hours, followed by recovery of the conditioned medium of the cells. After completion of recovery of the conditioned medium, the cells were counted using a cell counter.

### Example 2

### Isolation and Purification of Exosomes

To isolate exosome from the conditioned medium recovered in Example 1, ultracentrifugation was used. The recovered conditioned medium was subjected to sequential centrifugation at 4° C. to isolate exosomes as follows.

First, to remove cells from the recovered conditioned medium, the conditioned medium was centrifuged at 300×g for 10 minutes, and then the supernatant was collected. In addition, to remove cell debris from the supernatant, the supernatant was centrifuged at 2,000×g for 20 minutes, and then the supernatant was collected. Then, to remove microvesicles from the supernatant, the supernatant was centrifuged at 16,500×g for 10 minutes, and then the supernatant was collected. The finally obtained supernatant was centrifuged at 120,000×g for 120 minutes, and then the supernatant was discarded and the pellets were collected. Then, the pellets were washed with phosphate-buffered saline, and centrifuged again at 120,000×g for 120 minutes, and the supernatant was discarded. Next, the pellets were suspended in phosphate-buffered saline, thereby isolating exosomes.

Meanwhile, as methods of isolating exosomes from a conditioned medium of animal cells including stem cells, various methods known in the art may be used in addition to the isolation method as described above. For example, for isolation of exosomes, known isolation methods may be used, such as ultrafiltration, density gradient centrifugation, tangential flow filtration (TFF), size exclusion chromatography, ion exchange chromatography, immunoaffinity capture, microfluidics-based isolation, exosome precipitation, total exosome isolation kit, polymer based precipitation and the like. However, the method for isolating exosomes is not limited to the above-described methods, and it is of course possible to use various isolation methods that are being used in the art or may be used in the future.

### Example 3

### Characterization of Isolated Exosomes and Evaluation of Exosome Productivity

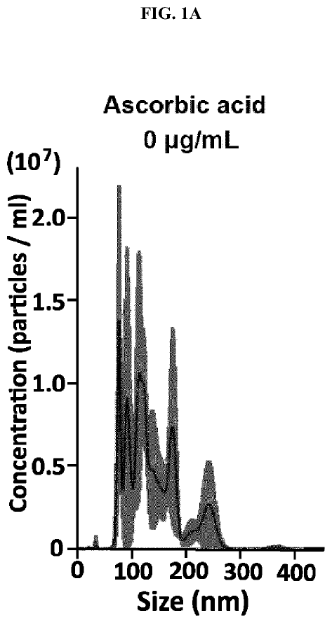

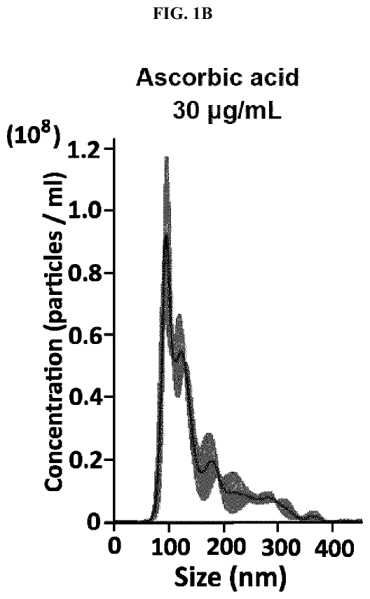

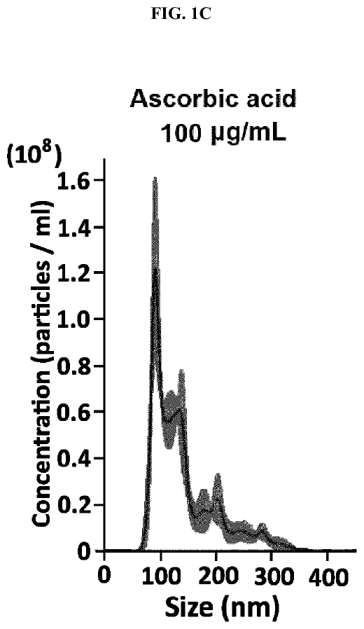

The particle size and concentration of the isolated exosomes were measured by nanoparticle tracking analysis (NTA) instrument (purchased from Malvern). FIG. 1 shows the results of NTA of the exosomes isolated by the isolation method according to one embodiment of the present invention. As shown in FIG. 1, it could be seen that the exosomes obtained after pre-treating and culturing the stem cells in the medium containing ascorbic acid according to one embodiment of the present invention (hereinafter referred to “experimental group 1”) had a more uniform particle size distribution than the exosomes obtained after culturing the stem cells in the medium not containing ascorbic acid (hereinafter referred to “experimental group 2”).

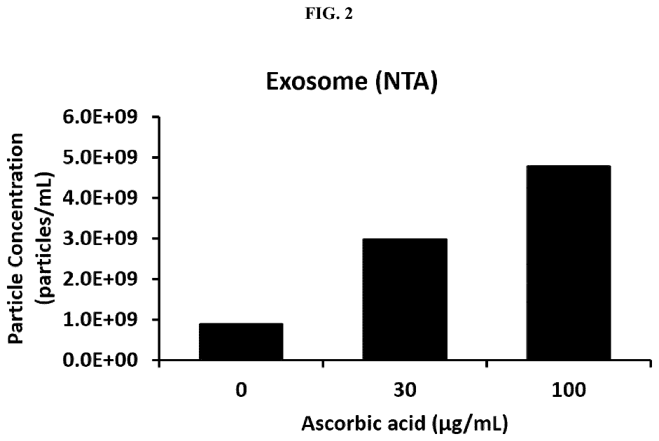

As shown in FIG. 2, from a comparative analysis result of NTA, it was confirmed that the productivity (i.e., the number of exosome particles per mL) of the exosomes derived from the stem cells pre-treated and cultured in the medium containing ascorbic acid (experimental group 1) increased by approximately four times or more as compared with the control group not treated with ascorbic acid (experimental group 2).

In addition, for accurate comparative analysis of exosome productivity, the exosomes of each of experimental groups 1 and 2 and Exosome-Human CD63 Flow Detection Reagent (Invitrogen™) were mixed together overnight. Then, each of the mixtures was allowed to react with PE mouse anti-Human CD63 (BD Parmigen™), and the PE mean fluorescence intensity (MFI) thereof was measured using flow cytometry.

Meanwhile, since CD63 is a representative positive marker of exosomes, the CD63 content and the exosome content are linearly proportional to each other, and an increase in the CD63 content means a proportional increase in the exosome content. According to this principle, human CD63 protein of known concentration was serially diluted to prepare human CD63 protein solutions having different human CD63 protein concentrations, and each human CD63 protein solution and Exosome-Human CD63 Flow Detection Reagent (Invitrogen™) were mixed together overnight. Then, each of the mixtures was allowed to react with PE mouse anti-human CD63 (BD Parmigen™), and the PE mean fluorescence intensity (MFI) thereof was measured using flow cytometry. Then, linear regression analysis was performed on the human CD63 protein concentration values and the corresponding MFI measurement values, and a standard quantitative analysis graph satisfying a linearity of 0.99 or higher was generated. Then, using the generated standard quantitative analysis graph, the CD63 content in each of experimental groups 1 and 2 was determined.

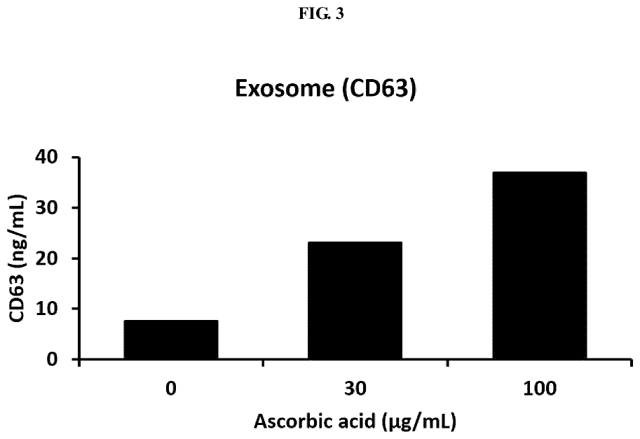

As a result, it was confirmed that the content of the exosomes (i.e., the content of CD63) derived from the stem cells pre-treated and cultured in the medium containing ascorbic acid remarkably increased compared to that in the untreated control group (see FIG. 3). From this result, it is understood that the present invention is able to enhance the productivity of exosomes secreted or released per cell (or cell-derived conditioned medium) by culturing cells in a medium containing ascorbic acid, an analogue thereof, or a derivative thereof.

Thus, the present invention is able to enhance the productivity of exosomes and/or extracellular vesicles secreted or released per cell (or cell-derived conditioned medium). Therefore, the present invention has advantages that it can economically and efficiently produce exosomes and/or extracellular vesicles, which can be utilized commercially and/or clinically, in high yield, and in particular, it can produce a large amount of exosomes and/or extracellular vesicles as compared with conventional methods.

Although the present invention has been described with reference to the embodiments, the scope of the present invention is not limited to these embodiments. Any person skilled in the art will appreciate that various modifications and changes are possible without departing from the spirit and scope of the present invention and these modifications and changes also fall within the scope of the present invention.

1. A method for promoting production of exosomes and/or extracellular vesicles, the method comprising culturing animal cells in a medium containing ascorbic acid, an analogue thereof, or a derivative thereof.

2. The method of claim 1, further comprising culturing the animal cells in a medium containing ascorbic acid, an analogue thereof, or a derivative thereof and fetal bovine serum (FBS).

3. The method of claim 2, wherein the fetal bovine serum is one from which FBS-derived exosomes and extracellular vesicles have been removed.

4. The method of claim 1, further comprising culturing the animal cells in a serum-free medium containing ascorbic acid, an analogue thereof, or a derivative thereof

5. The method of claim 1, wherein a concentration of the ascorbic acid, analogue thereof, or derivative thereof is 1 μg/mL to 300 μg/mL.

6. The method of claim 5, wherein the concentration of the ascorbic acid, analogue thereof, or derivative thereof is 10 μg/mL to 100 μg/mL.

7. A medium for promoting production of animal cell-derived exosomes and/or extracellular vesicles, the medium containing ascorbic acid, an analogue thereof, or a derivative thereof

8. The medium of claim 7, wherein a concentration of the ascorbic acid, analogue thereof, or derivative thereof is 1 μg/mL to 300 μg/mL.

9. The medium of claim 8, wherein the concentration of the ascorbic acid, analogue thereof, or derivative thereof is 10 μg/mL to 100 μg/mL.

10. The method of claim 2, wherein a concentration of the ascorbic acid, analogue thereof, or derivative thereof is 1 μg/mL to 300 μg/mL.

11. The method of claim 10, wherein the concentration of the ascorbic acid, analogue thereof, or derivative thereof is 10 μg/mL to 100 μg/mL.

12. The method of claim 3, wherein a concentration of the ascorbic acid, analogue thereof, or derivative thereof is 1 μg/mL to 300 μg/mL.

13. The method of claim 12, wherein the concentration of the ascorbic acid, analogue thereof, or derivative thereof is 10 μg/mL to 100 μg/mL.

14. The method of claim 4, wherein a concentration of the ascorbic acid, analogue thereof, or derivative thereof is 1 μg/mL to 300 μg/mL.

15. The method of claim 14, wherein the concentration of the ascorbic acid, analogue thereof, or derivative thereof is 10 μg/mL to 100 μg/mL.
